(19) United States
(12) Patent Application Publication
(10) Pub. No.: US 2025/0095853 A1
(43) Pub. Date: Mar. 20, 2025

(54) METHODS AND SYSTEMS FOR PROVISION OF AN OBSERVABLE INDICATING A MEDICAL DIAGNOSIS

(71) Applicant: Siemens Healthineers AG, Forchheim (DE)

(72) Inventors: Sven KOHLE, Erlangen (DE); Felix NENSA, Essen (DE)

(73) Assignee: Siemens Healthineers AG, Forchheim (DE)

(21) Appl. No.: 18/888,426

(22) Filed: Sep. 18, 2024

(30) Foreign Application Priority Data
Sep. 20, 2023 (DE) 10 2023 209 166.6

(51) Int. Cl. G16H 50/20; G16H 10/60; G16H 30/00

(52) U.S. Cl. CPC G16H 50/20; G16H 10/60; G16H 30/00

(57) ABSTRACT

A computer-implemented method for providing an observable indicating a medical diagnosis, comprises: obtaining an image data series of a patient, wherein the image data series has a number of medical image datasets, which have each been recorded over a period of time at different points in time; extracting a time series from the medical image data series; determining the observable based on the time series; and provisioning the observable.

### CROSS-REFERENCE TO RELATED APPLICATION(S)

The present application claims priority under 35 U.S.C. § 119 to German Patent Application No. 10 2023 209 166.6, filed Sep. 20, 2023, the entire contents of which is incorporated herein by reference.

### FIELD

Forms of embodiments of the present invention relate to methods and systems that are embodied, based on a time series analysis, to derive observables that can indicate a medical diagnosis. Forms of embodiments of the present invention further relate to methods and systems for deriving, selecting and comparing with one another or correlating such time series from raw data, such as medical image data or other patient data for example, in order to provide observables. Forms of embodiments of the present invention further relate to the use of the observables in the further clinical workflow, such as for example the creation of a medical diagnosis report, making a medical diagnosis in a (partly) automated manner or finding an option for treatment on the basis of the observables in each case.

### BACKGROUND

Time sequences play a decisive role in medical diagnostics. Thus it is frequently not the current value of a parameter that is decisive but also how this value has developed. Thus for example a shrinking lesion can have a completely different diagnostic indication from a growing one or one that has been detected for the first time.

A pertinent analysis is made more complicated however by individual values frequently being seen in the context of other values. Thus for example a patient's weight loss can be classified as a function or further values as non-critical or can point to a deterioration in the state of the patient. Formulated more generally for example an episode of an increased or reduced first value, which coincides with an episode of an increased or reduced second value, can point to an entirely different illness pattern than if the second value is constant.

One problem in the practical implementation of this knowledge lies in the fact that such information is frequently not accessible precisely during the diagnosis of medical image data. The doctor making the diagnosis therefore frequently limits himself or herself to considering image datasets obtained at different points in time alongside one another. Quantitative measured values are frequently not obtained. An inclusion of further time series outside radiology frequently fails due to lack of accessibility of such data and due to the limited time available for the diagnosis. Thus in clinical routine there is patently often not enough time to load individual documents from the patient file in order to check whether information relevant for the diagnosis is contained therein.

A further problem arises from the fact that data from various medical disciplines, such as radiology, laboratory or pathology is largely obtained and considered separately. With complex illnesses knowledge is actually gleaned from the various disciplines. This frequently occurs however in the form of individual presentations for the various aspects. This means that the radiologist reports on the radiology diagnosis, the pathologist about the pathology diagnosis and the internist about the symptoms of the patient. A systematic, i.e. quantitative and where necessary comparative analysis of the time sequences of individual values is made more difficult by this.

### SUMMARY

Against this background, an object of embodiments of the present invention is to provide methods and apparatuses that support a user in making the diagnosis of a patient and in particular make it easier to take account of what is happening over the course of time in clinical routine.

This and further objects are achieved with a method, a system, a computer program product or a computer-readable memory medium as claimed in the independent claim(s) and the dependent claims. Advantageous developments are specified in the dependent claims.

An inventive way in which at least the object is achieved will be described below both in relation to the claimed apparatuses and also in relation to the claimed method. Features, advantages or alternate forms of embodiment/aspects mentioned here are likewise also to be transferred to the other claimed subject matter and vice versa. In other words the physical claims (which are directed to an apparatus for example) can also be developed with features that are described or claimed in conjunction with a method. The corresponding functional features of the method can be embodied in this case by the corresponding physical modules.

In accordance with one aspect a computer-implemented method for providing an observable indicating a medical diagnosis is provided. The method has a number of steps. One step is directed to obtaining a series of image data of a patient, wherein the image data series has a number of medical image datasets that have been recorded over a period of time at various points in time in each case. A further step is directed to extraction of a time series from the medical image data series. A further step is directed to a determination of the observable based on the time series. A further step is directed to a provision of the observable.

Medical image datasets can generally relate to datasets that have been recorded with a medical imaging modality. A medical image dataset comprises image data that depicts a body, an area of the body or a part of the body of the patient. The medical image datasets of the image data series can each show the same body, the same area of the body or the same part of the body, but can have been recorded at different points in time.

Image data can relate to medical image data with two or three spatial dimensions. The medical image dataset comprises image data in the form of a two- or three-dimensional arrangement of pixels or voxels. Such arrays of pixels or voxels can be representative for color, intensity, absorption or other parameters as a function of the two- or three-dimensional position and can be obtained for example by suitable processing of measurement signals that have been obtained by a medical imaging modality.

Imaging modalities in this case can for example comprise computed tomography devices, magnetic resonance devices, x-ray devices, ultrasound devices and the like. Image data recorded with these or similar modalities is also referred to as radiology image data.

The medical image datasets can be formatted in a standard image format such as the Digital Imaging and Communications in Medicine (DICOM) format, corresponding to the DICOM PS3.1 2020c standard (or to a later or earlier version of this standard) for example. The medical image datasets can be stored in an electronic archiving or storage facility, such as a Picture Archiving and Communication system (PACS).

The period of time can be predetermined and can amount to weeks, months or days for example. In accordance with a few examples the period of time can be predetermined based context information described herein. An image data series has at least two, preferably more than two, image datasets, which have been recorded within the period of time.

Obtaining the image data series can, according to a few examples, comprise retrieval of the image datasets from an electronic memory facility. In particular this can comprise a retrieval of further image datasets based on an image dataset to be diagnosed. The image dataset to be diagnosed can in this case be the most recent image dataset of the image data series. The further image datasets can be chosen automatically from a number of image datasets of the patient in the memory facility for example. In accordance with a few examples this can be undertaken with the proviso that the further image datasets “match” the image dataset to be diagnosed in their content and/or timing, for example by them essentially showing the same body, the same area of the body or the same part of the body as the image dataset to be diagnosed or by having been recorded within the time period.

The time series can comprise a series of measured values that have been recorded at different points in time. In particular the measured values of the time series can be of a similar type. “Of a similar type” in this case can mean in particular that the measured values are measured values of the same measurement variable. In particular the time series can be a time series of a measurement variable associated with the patient (or the image data series of the patient). In particular the measurement variable can be a medical or physiological measurement variable, such as a size of an abnormality or a composition of the body of the patient. The measured values are based on the image datasets or are taken from them. In this case each measured value of the time series can correspond to or be taken from, especially precisely one, image dataset.

The measured values of the first time series can in particular be able to be derived from the image data of the image datasets. The measured values from the image datasets can be obtained or extracted in particular with image processing or image data analysis apparatus, device and/or means.

In accordance with a few examples the extraction step comprises an application of an image data analysis function to the medical image datasets of the medical image data series, wherein the image data analysis function is embodied to extract an image data-based measured value from a medical image dataset, and the time series has the image data-based measured values.

In accordance with a few examples the observable comprises a temporal behavior of the first time series or is based on a temporal behavior of the first time series. In particular the observable can be a measure for a temporal behavior of the first time series. In particular the observable can comprise an increase or a decrease in the first time series or be a measure thereof. Optionally the observable can comprise a temporal behavior of the first time series in proportion to one or more further time series, or be based thereon, or be a measure thereof.

In accordance with a few examples the provision of the observable comprises an output of the observable to a user. The user can for example be a doctor making the diagnosis, in particular a radiologist. An output can for example be undertaken via an, in particular graphical, user interface.

A provision of the observable can further comprise a provision of the observable to a further processing facility. The further processing can, based on the observables, comprise generating a result that is able to be evaluated for the user in the diagnostic process. For example this can comprise a medical examination report or parts of such a report or a recommendation for treatment.

The automated extraction of a time series from a series of medical image datasets mean firstly that the user is relieved of the work of comparing individual image datasets and of manually extracting measured values to be compared. Through the determination of the observables based thereon there is further an automated analysis of the time series to the extent of whether a medical diagnosis is indicated in the time series. Through this, the user is not only supported in the diagnosis of a patient, but taking time sequences into account in the clinical routine is made easier.

In accordance with one aspect, the step of determining the observable comprises obtaining a second time series of a measurement variable associated with the patient that differs from the time series, with said second time series covering a second period of time, which overlaps at least partly with the period of time, wherein the observables are additionally established based on the second time series.

The second time series can for example be extracted automatically from an electronic patient file or from patient data of the patient. The patient data in this case can comprise image data, in particular the image data series, and non-image data. As an alternative the second time series can also be chosen by a user from a choice of a number of available second time series and are obtained in this way. The available second time series, in accordance with a few examples can in particular be extracted automatically from patient data of the patient. Like the time series, the second time series can comprise a series of measured values that have been acquired at different points in time. In particular the measured values of the second time series can be of a similar type. “Of a similar type” in this case can mean in particular that the measured values are measured values of the same measurement variable.

The measurement variables of the second time series can be different from the measurement variables of the time series.

Taking account of the second time series enables various developments to be considered in relation to one another. This enables dependencies to be identified that are not accessible in an isolated consideration of individual measurement variables.

In accordance with a few examples the step of obtaining the second time series comprises an extraction of the second time series from the medical image data series.

The second time series then corresponds in its basic embodiment to the time series, but can in particular be based on a different measurement variable. In particular the explanations and definitions given herein for the time series can then also apply to the second time series. In particular an extraction of the second time series can comprise an application of a further image data analysis function to the medical image datasets of the medical image data series, wherein the further image data analysis function (differing from the image analysis function) is embodied to extract a further image data-based measured value (differing from the image data-based measured value of the time series) from a medical image dataset, and the time series has the further image data-based measured values.

This enables complementary information to be extracted from the image data and compared, which can improve the diagnostic validity of the observables.

In accordance with a few examples the step of obtaining the second time series comprises an extraction of the second time series from a second medical image data series different from the medical image data series.

The second image data series can have a number of medical image datasets, which have each been recorded over a second period of time at different points in time.

In accordance with a few examples the image datasets of the image data series can have been recorded by a first imaging modality and the image datasets of the second image data series can have been recorded by a second imaging modality that differs from the first imaging modality in its type of imaging. For example the first imaging modality can be a magnetic resonance modality, and the second imaging modality can be a computed tomography modality.

Referring back to different image data series enables complementary information to be extracted and compared, which can improve the diagnostic significance of the observables.

In accordance with one aspect the step of determining the observables comprises: a determination of correlation information based on the first time series and the second time series, wherein the correlation information specifies a measure for a temporal correlation between the first time series and the second time series, and establishing the observables based on the correlation information.

The correlation information can specify the temporal relationship between the time series and the second time series. Known correlation functions can be applied to the time series and the second time series for evaluation of the correlation information for example.

In accordance with a few examples the observable comprises a correlation of the time series with the second time series and in particular a correlation in the temporal behavior of the time series with the second time series or is based on such a correlation. In particular the observable can comprise a correlation or anticorrelation of the time series with the second time series or be a measure for it.

The evaluation of correlation information enables measurement variables to be analyzed systematically in proportion to one another. This enables dependencies to be quantified that are not accessible in an isolated consideration of individual measurement variables. Through this the user is able automatically to have information to hand in order to quickly come to an appropriate diagnosis of the patient.

In accordance with one aspect the measurement variable of the second time series is not based on medical image data.

In other words the second time series has not been extracted from image datasets. In particular the second time series can have been extracted from datasets of the patient that are not image data.

Non-image data is such patient data that is not image data. It can for example comprise one or more medical diagnosis reports, for example from radiology, pathology, the laboratory or further disciplines. The non-image data can in particular comprise longitudinal data, which contains or more medical values of the patient and/or elements from the illness history of the patient. In such cases it can involve laboratory data, vital values and/or other measured values or examinations relating to the patient.

The non-image data can for example be contained in an Electronic Medical Record, EMR, or Electronic Health Record, EHR. The electronic medical record in this case can be retrieved from one or more memory facilities, with said memory facilities being able to be linked into a medical information network. For example memory facilities can be interrogated for the non-image data or the electronic medical record of the patient. For this for example an electronic identifier, such as a patient ID or an access number can be used. Correspondingly the non-image data can be received from one or more of the available memory facilities, in which at least parts of electronic medical record are stored. The memory facilities in this case can for example be part of medical information systems, such as hospital information systems and/or PACS systems and/or laboratory information systems etc.

All available information of the patient can be stored in the electronic medical record, in particular non-image data, but also image data.

Taking account of a measurement variable that is not based on imaging enables further information outside the imaging diagnostics to be taken into account. The corresponding observable can thus relate various temporal developments to one another. This makes a better decision basis for a diagnosis possible for a user, without this entailing any significant extra effort for the user.

In accordance with one aspect obtaining the second time series comprises a retrieval of an electronic medical record of the patient and an extraction of the second time series from the electronic medical record.

In accordance with one aspect the method comprises an extraction of a number of time series from the image data series and/or obtaining a number of second time series, wherein in the step of determining for a number of different combinations (pairs) of time series selected from the time series and/or the second time series correlation information and an intermediate observable based thereon, based on the intermediate observables, individual combinations (pairs) are selected from time series, and the selected pairs are provided.

The selected combinations (pairs) can be displayed to the user by way of the provision (in a corresponding user interface for example). In particular the selected combinations (pairs) can be displayed together with the associated intermediate observables in order to provide an explanation for the selection. A selection can for example comprise a check to the extent of whether an intermediate observable exceeds a threshold value. A selection can further comprise a comparison with an observable pattern that indicates medically relevant subject matter.

By calculating correlations of a very widest diversity of combinations of time series (which can be undertaken in the background) there can be a systematic search for abnormalities that are relevant for the diagnostics. Such abnormalities can then be brought to the user's attention automatically. Consequently the user does not have to form and compare different time series manually, but has relevant correlations identified and displayed to them automatically.

In accordance with one aspect the measurement variable of the second time series comprises a laboratory value, in particular a blood value, of the patient.

Taking account of laboratory values enables data complementary to the imaging to be included automatically. In such cases it is shown that (small) abnormalities in the imaging frequently accompany changes in the laboratory or blood values. While observing individual values in isolation frequently does not indicate any illness, a comparison of these values can absolutely point to a significant illness.

In accordance with a few examples the measured values comprise an oxygen saturation, an ECOG score, an inflammation value, an alkaline phosphatase content (AP value) and/or an erythrocyte content in the patient's blood.

The oxygen saturation allows a statement to be made about the breathing or the efficiency of the oxygen transport and thereby about the constitution of the patient. A bad oxygen saturation can indicate further problems of the patient.

The ECOG score or performance status score describes the physical state of cancer patients and serves to quantify the overall wellbeing and the restrictions to activities of day-to-day life. On the basis of this value the progression of the illness can be estimated, suitable treatment determined, and a prognosis made.

Inflammation values, such as the C-Reactive Protein (CRP) value, give information about inflammations in the body. With infections, inflammations and tissue damage, the inflammation values are higher. A determination of the Alkaline Phosphatase (AP value) serves as an indicator for illnesses, such as those of the liver and the bile tracts, a tumor or for changes in the bone metabolism.

To many or too few erythrocytes in the blood can also indicate specific illnesses. For example a low value of erythrocytes can point to a disease of the bone marrow, for example with leukemia, or to tumor diseases.

The said values can therefor supplement the imaging well and help to better estimate measured values from the imaging.

In accordance with a few examples the measurement variable of the second time series comprises a medication of the patient.

A medication can comprise a type and amount of a medicament given to the patient.

Taking the medication into account enables it to be assessed for example whether a physical state of the patient from the imaging is caused by administering a medicament or not.

In accordance with a few examples the step of determining the observables comprises: obtaining a third time series of a further measurement variable associated with the patient that differs from the time series and the second time series, with said third time series covering a third period of time that overlaps at least partly with the first and second period of time, determination of correlation information based on the first time series, the second time series and the third time series, wherein the correlation information specifies a temporal correlation between the first time series, the second time series and the third time series, and a determination of the observables based on the correlation information.

Taking a third time series into account enables further information to be considered. In particular the measurement variable of the third time series can be a medication of the patient. This for example enables a measurement variable from the imaging (for example a physical property of the patient such as water retentions) to be considered in relation to a measurement variable from the laboratory (such as an AP value) and to a medication administered.

In accordance with one aspect the step of determining the observable comprises a step of obtaining a single event associated with the patient, which lies within the period of time, a step of determining correlation information based on the time series and the individual event, wherein the correlation information specifies a measure for the temporal correlation between the time series and the individual event, and a step of determining the observable based on the correlation information.

In accordance with one aspect the step of determining the observable comprises a step of obtaining an individual event associated with the patient, which lies within the second period of time, a step of determining correlation information based on the second time series and the individual event, wherein the correlation information specifies a measure for a temporal correlation between the second time series and the individual event, and a step of determining the observable based on the correlation information.

An individual event can for example relate to a therapy or a treatment of the patient. In particular the individual event can relate to a radiotherapy treatment, a surgical intervention, a chemotherapy treatment and/or an immunotherapy treatment.

An individual event can further relate to a state of health of the patient. In particular the individual event can comprise an accident, a seizure (such as a stroke), a start of an inflammatory disease and the like.

In accordance with a few examples the step of obtaining the individual event comprises an extraction of the individual event based on the time series in an electronic medical record or from patient data of the patient. In particular this can comprise searching for individual events in an electronic medical record or from patient data of the patient based on the period of time.

The correlation information can specify the temporal relationship between the time series, and in particular a temporal behavior of the time series, and the individual event. For evaluation of the correlation information for example a temporal behavior of the time series can be determined and related to extracted individual events.

The correlation information enables the time series to be related to possible individual events. This enables it to be determined automatically whether a temporal development in the time series coincides with an individual event or is linked to it temporally. Through this the user is not only supported in the diagnosis of a patient but taking account of time sequences in the clinical routine is made easier.

In accordance with one aspect the method further comprises a step of obtaining context information concerning a clinical picture of the patient, wherein the step of extraction comprises selecting an image data-based measurement variable from a number of different image data-based measurement variables based on the context information, and the first time series comprises image data-based measured values of the selected measurement variable.

The context information can in particular specify framework conditions that are relevant for a diagnostic activity of the user. Examples for framework conditions are: “diagnosis of a chest CT recording after a trauma”, “aftercare examination within the framework of a cancer therapy of organ X”, “analysis of an MR image of the lungs”, “confirmation of a suspected diagnosis Y” etc. The context information can be obtained for example based on a corresponding user input and/or the electronic medical record and/or an electronic work list.

Based on the context information a measurement variable that is especially relevant for the present case can be selected automatically. If for example a cancer patient is to be diagnosed starting from the context information, the proportion of muscle tissue as a measurement variable can provide information about a deterioration and an advance of the disease. Likewise a size of a lesion as a measurement variable can show whether a therapy is having an effect.

In accordance with a few examples, observables for measurement variables that have initially not been selected can be established in the background.

In accordance with a few examples the step of extraction further comprises selection of an image data analysis function from a number of image data analysis functions provided, wherein the selected image data analysis function is embodied to extract an image data-based measured value of the selected measurement variable from medical image datasets and an application of the selected image data analysis function to the medical image datasets of the medical image data series.

In accordance with a few examples the step of obtaining the second time series comprises a selection of a non-image data-based measurement variable from a number of different non-image data-based measurement variables based on the context information, and an extraction of non-image data-based measured values of the selected measurement variable from non-image data of the patient.

The selection of a measurement variable based on the context information automatically enables a measurement variable to be selected that is especially relevant for the present case. If for example a cancer patient is to be diagnosed, the AP value or the erythrocytes value can provide information about a progression of the disease.

In accordance with a few examples the step of obtaining the image data series of a patient comprises a selection of a number of image datasets for the image data series from a plurality of available image datasets of the patient based on the context information.

In accordance with a few examples, obtaining the second time series comprises retrieving an electronic medical record of the patient, selecting datasets from the medical record based on the context information and extracting the second time series from the selected datasets.

This for example, for a screening of a lung of the patient, explicitly enables image datasets or datasets to be selected from the medical record that image the region of the lungs or contain relevant information.

In accordance with a few examples the selection can be made based on rules, for example to the extent that a certain type of image datasets or datasets is always selected for a context. The datasets can for example be individual documents, such as reports or tables for example. In accordance with a few examples the selection can be made with the aid of metadata stored in the image datasets, such as the so-called DICOM tags, or with the aid of document titles or keywords in the documents.

In accordance with one aspect the medical image datasets each comprise a recording of an entire area of the body or a recording of the patient's entire body.

An area of the body can be region of the body of the patient, which comprises a number of body parts or organs. The region can for example be the thorax, chest, or the upper body region of the patient.

The use of image datasets that show an entire area of the body or even the entire body as a whole body recording, enables the time series to give information about the overall state of the patient, as is revealed by the imaging.

In particular the measurement variable of the time series can specify a state of the area of the body or of the entire body of the patient. This enables values in respect of the state of the body, which are only revealed with difficulty to users from a mere glance at the image data, to be quantified in the measurement variable.

In accordance with a few examples the measurement variable of the time series is selected from:
an analysis value of a body composition, BCA value, in particular comprising a proportion of muscle and/or a proportion of body fat and/or a proportion of water,
a size of a lesion,
a tumor load in an area of the body of the patient or in the entire body of the patient, and/or
a FDG distribution in an area of the body of the patient or in the entire body of the patient.

BCA stands for Body Composition Analysis and designates a method of extracting values from medical image data that characterize a general state of the body. They are thus not directed to individual lesions, but to overall characteristics of the body. Examples of this are the composition of muscle and fat tissue or an extent of water retentions. Changes in these values can for example indicate a progression of a cancer, but are only very difficult to read for the users themself from the image data. The reason for this is that, although human perception is trained to detect individual changes such as lesions, proportional estimations can be more difficult to make. This applies all the more since the human body involves a three-dimensional object. Consequently the image datasets are also three-dimensional image datasets, which can only be viewed overall with difficulty by a user in the two-dimensional screen view. The inventors have further recognized that in particular a correlation with blood values, such as the AP value or erythrocyte value make possible a well-founded idea about a progression of the course of a disease and represents a valuable observable.

In order to provide such BCA values, correspondingly embodied image analysis functions can be applied to the image datasets. In particular such image analysis functions can be embodied to provide a BCA value based on three-dimensional image datasets for the entire body or for an area of the body.

Detecting a size of lesion over a number of image datasets is likewise a challenge or is very time-consuming for the user. The lesion must be identified and measured by the user from image dataset to image dataset. Through an image analysis function, which is capable of tracking and measuring lesions from image dataset to image dataset, the user can be relieved of this work. A plurality of known detection algorithms is available for this purpose.

The tumor burden specifies how greatly the body or an area of the body of the patient is affected by tumors. Again this is a value which is only able to be estimated or even quantified by the user in its entirety with difficulty.

For example the tumor burden can be detected by administering suitable contrast or contrast means in a PET-CT examination with subsequent image data analysis by an appropriately embodied image data analysis function. The PET-CT examination is a combination of Positron Emission Tomography (PET) and Computed Tomography (CT). The PET can make metabolism processes in the body visible as images. For this purpose tiny amounts of radioactively marked substances are administered to the patient. The substances are distributed in the body and accumulate in specific tissue, for example tumors. In this the process can work with FDG (F-18 deoxyglucose) as the marker. FDG is a glucose module marked with radioactive fluorine. Since cancer cells have a higher consumption of glucose compared to healthy cells, there is an increase in accumulation of FDG in diseased cells.

The different distribution in the body cells is made visible with the aid of the PET camera. Even primary cancers measuring a few millimeters can be traced in this way.

The PET-CT combination device makes it possible to carry out a computed tomography almost simultaneously. Through the combination of the two methods cell regions with high glucose metabolism activity are able to be detected and in this way give information about the tumor burden.

The inventors have recognized that in particular a correlation of the tumor burden or of FDG values with blood values, such as the AP value or erythrocyte value makes it possible to provide well-founded information about a progression of the course of a disease and represents a valuable observable.

In accordance with a few examples the step of provision comprises a comparison of the observable with a predetermined observables pattern, and a display of the time series and/or of the observable based on the comparison.

If, in accordance with a few aspects and examples described herein, a correlation with a second time series is carried out the second time series can also be displayed accordingly together with the first.

The observables pattern can for example comprise one or more conditions for one or more observables, such as a threshold value for an anticorrelation of two measurement variables or for a decrease or increase in a measurement variable. An observables pattern can comprise a combination of a number of conditions for various observables. The observables pattern can be deemed to be fulfilled for example when all conditions are met.

The above selection can also be made when a number of time series or a number of second time series are present. Then only such time series or second time series are displayed for which the associated (intermediate) observable falls in an observables pattern (in that it lies above a threshold for example).

The selective display enables the user to be explicitly pointed to relevant observables. The display can therefore serve as a warning to the user about a deterioration in the clinical picture. At the same time the user is only informed when there are actually abnormalities, which relieves the load on the user.

In accordance with a few examples the method further comprises the steps of determining a medical diagnosis based on the observable, and a provision of the medical diagnosis.

A medical diagnosis in this case can comprise a prediction of a medical diagnosis, such as a confirmation or discarding of a suspected diagnosis. A medical diagnosis can further specify a probability for a presence of a medical diagnosis.

In accordance with a few examples the determination of the medical diagnosis can comprise a selection of the medical diagnosis from a number of suspected diagnoses.

The number of suspected diagnoses can be provided for example based on the context information. They represent a preselection from which the diagnosis that, based on the observables, best matches the case can be selected. In this case the suspected diagnoses can each be associated with one or more indicators, which show the suspected diagnosis. The diagnosis can thus be determined based on a comparison between the observable and the indicators.

If a number of observables are present (for example for a number of (second) time series) all can be selected for a determination of the medical diagnosis, or a selection from a number of suspected diagnoses, or a comparison with the indicators can be included.

With provision of the diagnosis the user, based on the observable, a suggestion for a possible diagnosis is offered. In other words a diagnosis is predicted. Through this the user is effectively supported in the diagnosis of a patient without time series having to be manually produced and evaluated.

In accordance with a few examples the provision of the medical diagnosis comprises a creation of a medical diagnosis report based on (and/or using) the observable and/or the diagnosis.

For example the diagnosis can be transferred into a template for a medical examination report, facultatively together with the observable and/or the time series or the second time series. In particular there can also be provision for selecting the template based on the observable (and/or diagnosis) from a number of available templates.

A medical diagnosis report can be a result of a diagnostic process that aims to determine the state of a patient with respect to one or more clinical aspects based on the medical data relevant for this. A medical examination report can have a document. In particular the medical examination report can have a structured document or structured document parts. A medical examination report can further have an unstructured document or unstructured document parts. The medical examination report can further be constructed from one or more templates, which will enter patient-specific information within the framework of a diagnosis.

The provision of a medical diagnosis report enables the user to be provided with a processing report that can be directly further used.

In accordance with one aspect the step of determining the medical diagnosis comprises a selection of a disease pattern from a selection of a number of disease patterns based on the observable, a retrieval of an electronic medical record of the patient from a database, a reconciliation of the selected disease pattern with entries in the electronic medical record, and a determination of the medical diagnosis based on the reconciliation.

A disease pattern can have one or more characteristics or indicators that show a disease. The characteristics or indicators can for example be ranges of values for a measurement variable and/or an observable that is based on one or more measurement variables. The measurement variables can be image data-based measurement variables or further, non-image data-based measurement variables.

Disease selection patterns can be predetermined. Disease selection patterns can further be preselected from a plurality of available disease patterns based on the context information.

Disease selection patterns can further be extracted from electronic compendia or textbooks. An example of such an electronic textbook is eRef by Thieme. A disease pattern can be extracted for example with a so-called Large Language Model, LLM, as described herein. Disease selection patterns can further be obtained based on disease patterns of comparable patients.

A reconciliation can comprise searching for information in the electronic medical record and a comparison with the disease pattern. In particular the electronic medical record can be searched for characteristics or indicators from the disease pattern. A search can further be made in the electronic medical record for measurement variables and/or observables and the associated values can be compared with ranges of values in the disease pattern.

The use of disease patterns enables a more accurate prediction of a diagnosis to be made, since it does not have to be just based on the observable. Through this the user obtains a reliable and easy-to-assess output.

In accordance with a few examples the reconciliation comprises establishing that an observable and/or measurement variable is missing in the electronic medical record by comparison with the disease pattern and, optionally, an application of an analysis function to the electronic medical record for provision of the missing observable and/or measurement variable and/or creation of an examination protocol for an examination of the patient for providing the missing observables and/or measurement variable.

The examination protocol can comprise general recommendations or instructions for the user or for other personnel involved as to how to carry out an examination. The examination protocol can further comprise control commands for activating a modality for carrying out the examination. The examination can for example be an imaging examination with an imaging modality.

Establishing missing measurement variables on the one hand enables information to be provided about how reliable the diagnosis provided is. On the other hand the user is given information about how they can further safeguard a diagnosis. The optional automated application of an analysis function or the optional automated creation of an examination protocol, building on this, can further relieve the load on the user, since the next steps for safeguarding the diagnosis are initiated automatically.

In accordance with a few examples a reconciliation of the selected disease pattern with the electronic medical record can be undertaken using a transformer network and in particular a Large Language Model, LLM.

A transformer network is a neural network architecture that generally has an encoder, a decoder or an encoder and a decoder. In a few cases the encoder and/or decoder consist of a number of corresponding encoding layers or decoding layers. Located within each encoding and decoding layer is a so-called attention mechanism.

The attention mechanism, often also referred to as self-awareness, relates data elements (for example words) within a series of data elements to other data elements within the series. The self-awareness mechanism makes it possible for the model for example to examine a group of words within a sentence and to determine the meaning that other groups of words within this sentence have for the group of words being examined.

In particular the encoder can be configured so that it converts the input (an element of the disease pattern or a document in the electronic medical record) into a numerical representation. The numerical representation can comprise one vector per input token (per word for example). The encoder can be configured so that it implements an attention mechanism, so that each vector of a token is influenced by the other tokens in the input. In this way the tokens can be related to one another and for example also be identified as relevant for the disease pattern when no word-for-word correspondence is present.

The transformer network can further comprise a classification module or a classification unit, which is configured so that it assigns to the output of the encoder or decoder a set of learned outputs, such as an element of the disease pattern for example.

The training of a transformer model, according to a few examples, can take place in two stages, a pre-training and a fine training. In the pre-training phase a transformer network can be trained with a large body of data, in order to learn the underlying semantics of the problem. Such pre-trained transformer networks are available for various languages. For specific applications described herein a fine training can be undertaken based on medical texts with verified (annotated) meanings and/or medical ontologies such as RADLEX and/or SNOMED. This enables the transformer network to learn typical relationships and synonyms of medical expressions.

For further information about transformer networks the reader is referred to the publication “Attention Is All You Need”, in arXiv: 1706.03762, Jun. 12, 2017, the contents of which are included here by reference in their entirety.

An LLM can be based on a transformer network. An LLM is a language model that is characterized by its size. The artificial neural networks with which it is constructed can contain dozens of millions up to billions of weights and are pre-trained with self-supervised learning and semi-supervised learning. Transformer networks make a decisive contribution to faster learning. Examples of LLMs, which can be used to reconcile the disease pattern with the electronic medical record are the GPT series (GPT-3.5 and GPT-4; used in ChatGPT), the LLaMa series, PaLM (used in Google Bard), BLOOM, Ernie 3.0 Titan or Claude.

An advantage of transformer networks lies in the fact that, due to the attention mechanism, wide-ranging dependencies in the input data can efficiently be taken into account. Over and above this encoders are capable of processing data in parallel, which saves on processing resources. Over and above this decoders are capable, due to their autoregression, of iteratively generating a sequence of output values with great certainty.

In accordance with one aspect the method comprises the steps: Comparison of the observables with corresponding observables of a number of reference patients different from the patient, selection of a comparison patient from the number of reference patients based on the step of comparison, provision of information about the comparison patients as comparison information.

In accordance with a few examples the information about the comparison patient can comprise an electronic medical record of the comparison patient or an extract from the electronic medical record, such as a diagnosis report for example.

Electronic patient records of the reference patients can be stored for example in a memory facility of a medical Information system. The corresponding observable of the respective patient can be contained in the electronic medical record in each case. In accordance with a few examples corresponding observables can be obtained using a transformer network, in particular an LLM.

In accordance with one aspect the method further comprises a step of selecting a comparison patient from a number of reference patients based on the observable.

In accordance with a few examples the comparison patient can be selected based on the selected disease pattern. In accordance with a few examples the selection can comprise a reconciliation of the selected disease pattern with the electronic medical record of the reference patient in each case.

In accordance with one aspect, for each corresponding observable, the step of comparison comprises determining a measure of similarity with the observable, wherein in the selection step a selection is made with the aid of the measure of similarity.

In accordance with one aspect the provision of the comparison information comprises an output of a warning if a critical state is indicated for the comparison patient.

The automated selection of comparison patient enables similar cases/patients to be brought to the user's attention. The user can then orient themselves to the similar cases in the diagnosis and the selection of treatment options. Through this the user can be effectively supported in the diagnosis and treatment of the patient.

In accordance with a few examples the method further comprises the steps of determining a treatment option based on the observable and of providing the treatment option.

In accordance with a few examples the step of determining a treatment option comprises selection of a treatment option from a selection of a number of treatment options based on the observable.

In accordance with a few examples the step of selecting the treatment option is additionally based on the context information and/or the selected disease pattern and/or the medical diagnosis determined.

In accordance with a few examples the method further comprises a pre-selection of the treatment option selected from a plurality of treatment options based on the context information and/or the disease pattern selected and/or the medical diagnosis determined.

In accordance with a few examples the step of determining a treatment option further comprises obtaining an Indication pattern for the treatment option selected, a retrieval of an electronic medical record of the patient from a database, reconciliation of the indication pattern with the entries in the electronic medical record, and a verification of the treatment option based on the reconciliation.

An indication pattern can comprise indications and/or contraindications for a treatment option. The indication pattern can be associated with the selected treatment option, for example based on one or more medical guidelines.

In accordance with a few examples the reconciliation of the indication pattern comprises ascertaining that an observable and/or measurement variable is missing in the electronic medical record by comparison with the indication pattern and, optionally, application of an analysis function to the electronic medical record for provision of the missing observable and/or measurement variable and/or creation of an examination protocol for an examination of the patient for provision of the missing observable and/or measurement variable.

In accordance with one aspect a system for provision of an observable indicating a medical diagnosis is provided. The system has a computing facility and an interface. The interface is embodied to receive an image data series of a patient, wherein the image data series has a number of medical image datasets, which have each been recorded at different points in time over a period of time. The computing facility is embodied to extract a time series from the medical image data series. The computing facility is further embodied to determine the observable based on the time series. The computing facility is further embodied to provide the observable via the interface.

The computing facility can be embodied as a back-end computing facility. In particular the computing facility can be embodied as a server system. The computing facility can have a cluster or a group of computing facilities and data memories. The back-end computing facility can be connected for data transfer to a front-end computing facility via a medical information network, with said front-end-computing facility hosting a user interface for the user. The computing facility can be connected for data transfer to a number of different (but in particular similar) front-end computing faculties via the medical network. The front-end computing facility (facilities) can belong to a medical organization, such as a practice for example, a hospital or a hospital network. The back-end computing facility can likewise belong to the medical organization or be embodied outside the medical organization. The back-end computing facility can be connected for data transfer via the medical information network to a number of different front-end computing facilities, which each belong to different medical organizations.

As an alternative the computing facility can also be distributed over the back-end and the front-end computing facility or only comprise the front-end computing facility.

In accordance with forms of embodiment the medical information network can be based on the HL7 standard. Health Level 7 (HL7) is a group of international standards for the exchange of data between organizations in healthcare and their computer systems. In particular communications and/or data connections can be based on the FHIR standard. Fast Healthcare Interoperability Resources (FHIR) is a standard formulated by HL7. It supports the exchange of data between software systems in healthcare. The use of the HL7 or FHIR standard enables data to be transmitted in structured form and no reformatting is necessary.

The interface can generally be embodied for exchange of data between the computing facility and further components. The interface can be implemented in the form of one or a number of individual data interfaces, which can have a hardware and/or software interface, for example a PCI bus, a USB interface, a FireWire interface, a ZigBee or a Bluetooth interface. The interface can further have an interface of a communication network, wherein the communication network can have a Local Area Network (LAN), for example an Intranet or a Wide Area Network (WAN) or an Internet. Accordingly the one or the number of data interfaces can have a LAN interface or a Wireless LAN interface (WLAN or Wi-Fi).

The system can further have a data memory connected to the interface, in which the image datasets and/or electronic medical records and/or analysis function and/or image data analysis functions and/or a transformer network/LLM are stored and are able to be called up via the interface and are thus able to be provided. The data memory can be embodied in such cases as a central or decentral memory unit or as Cloud storage.

The system can further comprise one or more imaging modalities, such as for example a computed tomography system, a magnetic resonance system, an angiography system, an x-ray system, a Positron Emission Tomography system, a mammography system, and/or such like.

The advantages of the proposed systems essentially correspond to the advantages of the proposed methods. Features, advantages or alternate forms of embodiment/aspects of the method can likewise be transferred to the other claimed subject matter and vice versa.

In a further aspect, an embodiment of the present invention relates to a computer program product, which comprises a program and is able to be loaded directly into a memory of a programmable controller and has computer-executable instructions, a computer program and/or program means, for example libraries and auxiliary functions for providing a method for provision of an observable indicating a medical diagnosis in particular for carrying out the method in accordance with the aforementioned forms of embodiment/aspects, when the computer program product is executed.

Embodiments of the present invention further relates, in a further aspect, to a computer-readable memory medium, on which readable and executable program sections are stored for carrying out all steps of a method for provision of an observable indicating a medical diagnosis in accordance with the aforesaid forms of embodiment/aspects when the program sections are executed by the controller.

The computer program products can in such cases be software with a source code that still has to be compiled and linked, or that just has to be interpreted, or comprises an executable software code, which for execution only has to be loaded into the processing unit. The method can be carried out quickly, identically repeatably and robustly via the computer program products. The computer program products are configured so that, via the processing unit, they can carry out the inventive method steps. The processing unit must in this case have the requirements such as for example a corresponding RAM, a corresponding processor, a corresponding graphics card or a corresponding logic unit, so that the respective method steps can be carried out efficiently.

The computer program products are stored for example on a computer-readable memory medium or are held in a network or on a server, from where they can be loaded into the processor of the respective processing unit, which can be directly connected to the processing unit or can be embodied as part of the processing unit. Furthermore control information of the computer program products can be stored on a computer-readable memory medium. The control information of the computer-readable memory medium can be embodied in such a way that, when the data medium is used in a processing unit, it carries out an inventive method. Examples of computer-readable memory media are a DVD, a magnetic tape or a USB stick, on which electronically readable control information, in particular software, is stored. When this control information is read from the data medium and stored in a processing unit, all inventive forms of embodiment/aspects of the method described above can be carried out. In this way, embodiments of the present invention can also be based on the said computer-readable medium and/or the said computer-readable memory medium. The advantages of the proposed computer program products or of the associated computer-readable media essentially correspond to the advantages of the proposed methods.

### BRIEF DESCRIPTION OF THE DRAWINGS

Further special features and advantages of the present invention will become evident from the explanations of exemplary embodiments with the aid of schematic diagrams. Modifications mentioned in this connection can be combined with one another in each case in order to form new forms of embodiment. The same reference characters are used for the same features in different figures.

In the figures:

FIG. 1 shows a schematic diagram of a form of embodiment of a system for provision of an observable indicating a medical diagnosis,

FIG. 2 shows a flow diagram of a method for provision of an observable indicating a medical diagnosis in accordance with one form of embodiment,

FIG. 3 shows a diagram of optional method steps in a method for provision of an observable indicating a medical diagnosis in accordance with one form of embodiment,

FIG. 4 shows a data flow diagram of a method for provision of an observable indicating a medical diagnosis in accordance with one form of embodiment,

FIG. 5 shows a schematic diagram of a graphical user interface in a method for provision of an observable indicating a medical diagnosis,

FIG. 6 shows a diagram of optional method steps in a method for provision of an observable indicating a medical diagnosis in accordance with one form of embodiment,

FIG. 7 shows a diagram of optional method steps in a method for provision of an observable indicating a medical diagnosis in accordance with one form of embodiment,

FIG. 8 shows a diagram of optional method steps in a method for provision of an observable indicating a medical diagnosis in accordance with one form of embodiment, and

FIG. 9 shows a diagram of optional method steps in a method for provision of an observable indicating a medical diagnosis in accordance with one form of embodiment.

### DETAILED DESCRIPTION

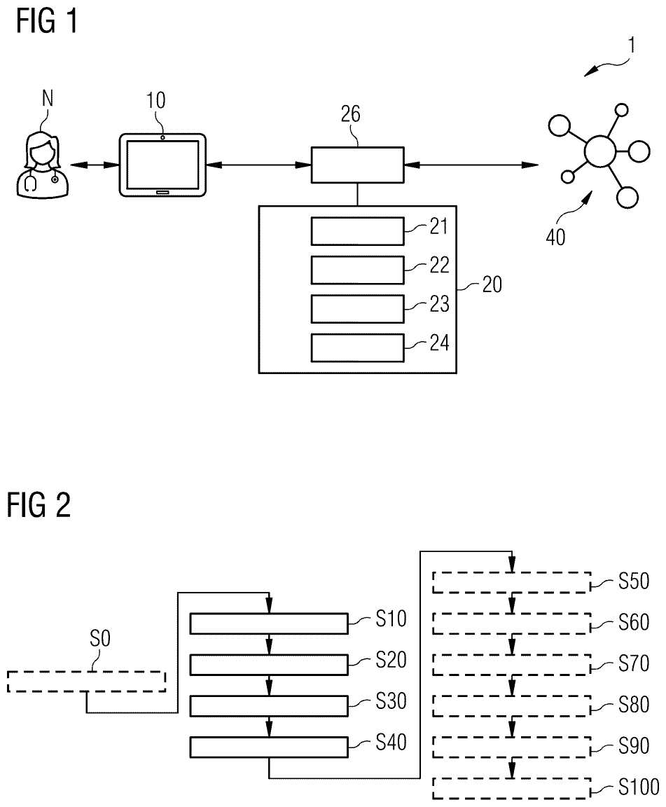

Shown in FIG. 1 is a system 1 for provision of an observable OBS indicating a medical diagnosis in accordance with a one form of embodiment. The system 1 has at least a front-end computing facility 10 with a user interface 10 (or also just user interface 10), a medical information system 40 and a back-end computing facility 20 (or also just computing facility 20), which are connected to one another for communication via a medical network or a data interface 26.

The medical image datasets SET and further information, such as for example electronic medical records EPA can be provided to the front-end computing facility 10 via suitable interfaces 26 by the medical information system 40. Typically a system such as the one shown in FIG. 1 has a plurality of front-end computing facilities 10, which all access the same medical information system 40 or are exchanging data with the back-end computing facility 20. In the form of embodiment shown the back-end computing facility 20, the medical information system 40 and the front-end computing facility (facilities) 10 are part of the same medical organization. A medical organization can for example be a practice, a group of practices, a hospital or a group of hospitals. Accordingly the network connecting these components via the interface 26 can be embodied as an internal network of the organization and comprise an intranet for example (such as a Local Area Network and/or a Wireless Local Area Network).

The front-end computing facility 10 can for example be embodied as a diagnostics center or diagnostics workstation, at which a user N can view and analyze electronic patient data of an electronic medical record EPA, or a medical image dataset SET and also can create, check, change, appraise and define medical diagnoses DIG and treatment options BHO. The front-end computing facility 10 can therefore also be referred to as the user interface 10. The front-end computing facility 10 can have a physical user interface, for example comprising a display and/or an input facility. The front-end computing facility 10 can have a processor. The processor can have a Central Processing Unit (CPU), a Graphics Processing Unit (GPU), a Digital Signal Processor (DSP), an image processing processor, an integrated (digital or analog) circuit or combinations of the aforementioned components and further facilities for providing an observable OBS, a diagnosis DIG and/or a treatment option BHO in accordance with forms of embodiment. The front-end computing facility 10 can for example comprise a desktop PC, laptop or a tablet.

The medical information system 40 can generally be embodied for acquiring and/or storing and/or forwarding medical image datasets SET and electronic medical records EPA. For example the medical information system 40 can have one or more databases (not shown). In particular the databases can be realized in the form of one or more Cloud storage modules. As an alternative the databases can be realized as local or distributed storage, for example as a PACS (Picture Archiving and Communication system), a hospital information system (KIS) a laboratory information system (LIS), an Electronic Medical Record (EMR) information system and/or further medical information systems. In accordance with a few examples the medical information system 40 can for example also comprise one or more medical imaging modalities (not shown), such as a computed tomography system, a magnetic resonance system, an angiography system, a C-arm x-ray system, a positron emission tomography system, an x-ray system or the like.

The medical image dataset SET can have medical image data. Image data can also relate in this connection to medical image data with two or three spatial dimensions. The image data can have been created with an imaging medical modality for example, such as for example an X-ray, computed tomography, magnetic resonance, positron emission tomography or angiography device or further devices. Such image data can also be referred to as radiology image data.

The image data contained in the medical image dataset SET can be formatted for example according to the DICOM format. DICOM (Digital Imaging and Communications in Medicine) is an open standard for the communication and administration of medical image data and associated data.

The image datasets SET of a patient can be contained in the electronic medical record EPA of the patient.

The electronic medical record EPA can also comprise non-image data as well as medical image datasets SET. Non-image data can for example be examination results that are not based on medical imaging. These can comprise laboratory data, vital data, spirometry data or protocols of neurological examinations. As well as this, non-image data can comprise text datasets, such as structured and unstructured medical reports. Non-image data can further also be patient-related data. This can for example comprise demographic information about the patient, such as data concerning their age, their gender or their body weight. The non-image data can be linked into the image data as metadata for example. As an alternative or in addition the non-image data can also be held in separate documents.

The back-end computing facility 20 can have a processor. The processor can be a Central Processing Unit (CPU), a Graphics Processing Unit (GPU), a Digital Signal Processor (DSP), an image processing processor, an integrated (digital or analog) circuit, or combinations of the aforementioned components and further facilities for provision of an observable OBS in accordance with forms of embodiment. The back-end computing facility 20 can be implemented as individual components or have a group of processors, such as a cluster. Such a system can be called a server system. Depending on form of embodiment, the back-end computing facility 20 can be embodied as a local server. The back-end computing facility 20 can further have a working memory, such as a RAM, in order for example temporarily to store image datasets SET, electronic medical records EPA or time series ZR1, ZR2. The back-end computing facility 20 is embodied by computer-readable instructions, by design and/or hardware in such a way that it can carry out one or more method steps in accordance with forms of embodiment of the present invention.

The back-end computing facility 20 can be connected via the interface 26 to the front-end computing facility 10 and/or the memory facility RD and/or the medical information system 40. Via this interface 26 the back-end computing facility 20 can receive medical image datasets SET, electronic medical records EPA and/or user inputs INP, on the basis of which time series ZR1, ZR2 can be automatically selected and observables OBS created and provided with computer assistance.

For provision of an observable OBS the back-end computing facility 20 can have a number of modules available to it.

Module 21 is embodied as a data retrieval module. It can be embodied to access the medical information system 40 and search for the medical image datasets SET or for electronic medical record EPA. In particular the module 21 can be embodied to formulate search queries and to pass them to the medical information system 40.

Module 22 can be embodied as a user interaction module or unit. The module 22 can be embodied to provide the user N with time series ZR1, ZR2 and observables OBS, and also, building on this, diagnoses DIG and/or treatment options BHO. The module 22 can further be embodied to detect one or more user inputs INP and to provide processing in the back-end computing facility 20. Such user inputs INP can for example comprise speech, gestures, eye movements, handling of input devices such as a computer mouse etc. for example. The user inputs INP can be directed to an interaction with the medical image dataset SET.

Module 23 can be regarded as an analysis module. Module 23 is embodied for extraction of the time series ZR1, ZR2 and for their analysis. For example module 23 can be embodied to extract time series ZR1 from image datasets SET. Such time series ZR1 are time series of an image data-based measurement variable BD-MG. Module 23 can further be embodied to extract time series ZR2 from electronic medical records EPA that relate to a non-image data-based measurement variable NBD-MG. Module 23 can be embodied to carry out corresponding analysis functions AF. For example one or more such analysis functions AF can be embodied to extract measured values for an image data-based measurement variable BD-MG from image datasets SET by image processing (image data analysis function). One or more analysis functions AF can further be embodied to extract from electronic medical records EPA measured values for a non-image data-based measurement variable NBD-MG.

For analysis of the extracted time series ZR1, ZR2 and for provision of the observable OBS the module 23 can further be embodied to carry out a time series analysis function ZR-AF. The time series analysis function ZR-AF is then embodied to establish a temporal development of one or more time series ZR1, ZR2. In particular the time series analysis function ZR-AF can comprise a correlation algorithm, which is embodied to establish correlation information KOR between two time series ZR1, ZR2, on the basis of which the observable OBS can be provided.

The module 24 can be understood as a module that goes further or LLM module. It can be embodied to convert the observable OBS provided by module 23 into results that can be usable for the user N. For example module 24 can be embodied to provide the observable OBS as text in natural language. Module 24 can further be embodied, based on the observable OBS—optionally with further reconciliation with the electronic medical record EPA—to provide a diagnosis DIG and/or a treatment option BHO. Module 24 can further be embodied, based on the observable OBS, to search in the medical information system 40 for comparison patients that have a similar case constellation to the patient.

Module 24 can be embodied to host and to execute a transformer network or Large Language Model (LLM). The LLM can be used to transfer the observable OBS into natural language and/or to carry out a reconciliation with electronic medical records EPA.

The division undertaken of the back-end computing facility 20 into modules 21-24 merely serves in this case to simplify the explanation of the way in which the back-end computing facility 20 functions and is not to be understood as being restrictive. The modules 21-24 or their functions can also be grouped together in one element. The modules 21-24 in this case can in particular also be interpreted as computer program products or computer program segments that, when executed in the back-end computing facility 20, realized one or more of the method steps described herein.

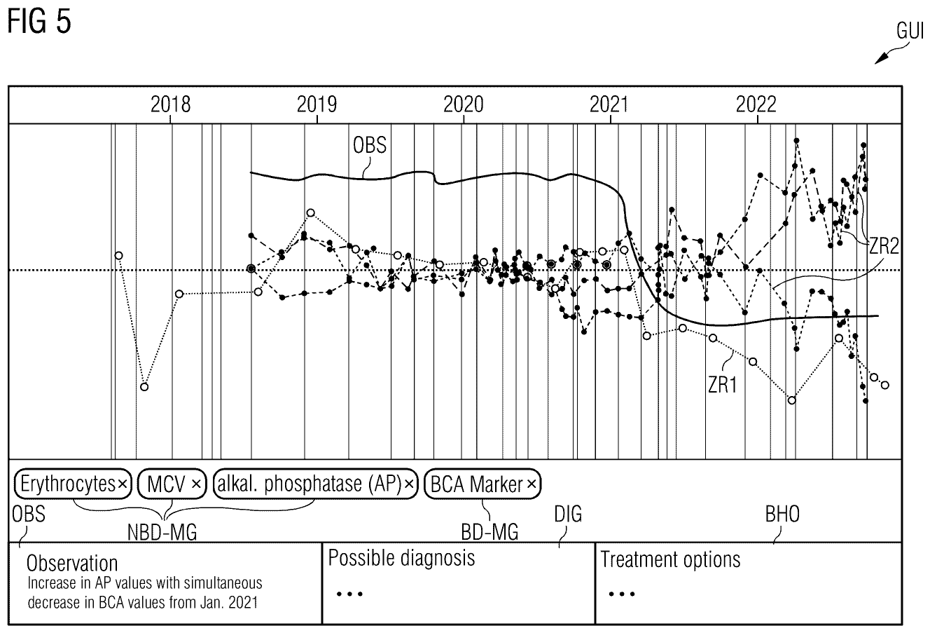

Shown in FIG. 2 is a schematic flow diagram of a method for provision of an observable OBS. The order of the method steps is not restricted either by the order shown or by the numbering selected. Thus the order of the steps can be changed where necessary and individual steps can be left out. Moreover one or more steps, in particular a sequence of steps, and optionally the entire method, can be carried out repeatedly. Shown in FIG. 4 is an associated diagram, which shows examples of the data flows associated with the method shown in FIG. 2. Unless specified otherwise, optional steps and data flows in FIGS. 2 and 4 are shown by broken lines.

First of all, in an optional step S0, context information KI can be obtained. The context information KI comprises a medical context for the patient to be diagnosed. This can for example be a diagnostic task, which the user N has to deal with with the aid of the available patient data. In addition or as an alternative the context information KI can comprise a specification of a clinical state of the patient. In addition or as an alternative the context information KI can comprise a specification of a possible disease or diagnosis of the patient.

The context information KI can for example be derived from an electronic medical record EPA of the patient. In addition or as an alternative inputs of the user via the user interface 10 can be taken into account for the context information KI.

In step S10 an image data series SE of the patient is obtained. The image data series SE has a number of individual image datasets SET of the patient that have been recorded at different times. The image datasets SET can for example be loaded in step S10 from the medical information system 40. In this case the context information KI can optionally be taken into account, so that only such image datasets SET are loaded that correspond to the context information KI. The loading of the image data series SE can be initiated for example by a manual selection of the respective case/patient by the user N via the user interface 10.

In step S20 one or more time series ZR1 is or are extracted from the image datasets SET. These time series ZR1 are also called first time series ZR1 or also image data-based time series ZR1. The time series ZR1 have a longitudinal series of measured values for a measurement variable BD-MG. The measurement variables BD-BG can be taken in step S20 from the image datasets, for example by application of an appropriate image analysis function AF to the image datasets SET.

The measurement variable(s) BD-MG can be predetermined. As an alternative measurement variables BD-MG based on a number of basically possible measurement variables BD-MG can be specifically selected for the respective patient. This can be undertaken for example based on the context information KI.

The first time series ZR1 basically have measured values that can be extracted from medical image datasets SET, i.e. image data. BCA values such as a proportion of body fat, a proportion of muscle etc. could be mentioned by way of example at this point, such as are produced from scans of the patient's body.

In step S30 an observable OBS is established from the first time series or number of time series ZR1, which is relevant for a diagnosis or treatment of the patient or shows a diagnosis or treatment. In particular the observable OBS can comprise a temporal development of one or more time series ZR1, ZR2. For example the observable OBS can comprise a temporal derivation of a time series ZR1, ZR2.

In step S40 the observable OBS is provided. This can comprise a display of the observable OBS in the user interface 10. Optionally underlying time series ZR1, ZR2 can be shown here.

The provision in step S40 can further comprise passing on the observable for optional further processing. A few possible further processing steps are listed below with reference to steps S50-S100. The steps are optional and can follow on individually or in any given combination from step S40.

In optional step S50, based on the observable OBS, a medical diagnosis DIG can be predicted. Thus for example a temporal development of a time series ZR1, ZR2 (either on its own or in relation to other time series ZR1, ZR2) can indicate a medical diagnosis DIG. Optionally, in step S50, in the prediction of the medical diagnosis DIG, further information such as the context information KI or information from the electronic medical record EPA of the patient can be taken into account.

The prediction of the medical diagnosis DIG can be a rule-based prediction. As an alternative the prediction is determined in step S50 by application of a trained algorithm to the observable OBS, optionally to the context information KI and/or to the electronic medical record EPA.

In step S60 the diagnosis DIG is provided. This can comprise a display of the diagnosis DIG in the user interface 10. Optionally underlying time series ZR1, ZR2 and also further information of significance for the decision can be shown here.

In step S70, based on the observable OBS, one or more comparison patients can be determined who have a medical similarity to the patient. To this end a number of reference patients can be provided in step S70 from whom, with the aid of the observable OBS, comparison patients who have an increased similarity to the patient can be selected (based on the observables OBS).

In addition, in the selection of the comparison patient, further information such as the context information KI or information from the electronic medical records EPA of the patient/of the reference patients can also be taken into account.

In step S80 information VI for the comparison patients can then be provided (also called comparison information VI). For example comparison information VI can be extracted from the electronic medical record EPA of the comparison patients. The comparison information VI can for example comprise a diagnosis, a treatment carried out, a course of the disease and the like. As an alternative the comparison information VI can comprise the respective electronic medical records EPA of the comparison patients or parts thereof.

The comparison information VI can be provided to the user N by displaying it in the user interface 10. For example the user N can be provided with a link to the electronic medical records EPA of the comparison patients.

In step S90, based on the observable OBS, a treatment option BHO for the patient can be determined. In this way for example a temporal development of a time series ZR1, ZR2 can indicate a treatment option BHO. Optionally in step S90, in the determination of the treatment option BHO, further information such as the medical diagnosis DIG, the context information KI or information from the electronic medical record EPA of the patient can be taken into account. In a way similar to prediction of the medical diagnosis DIG, the treatment option BHO can be rule-based or can be determined using a trained algorithm.

In step S100 the treatment option BHO is provided. This can comprise a display of the treatment option BHO in the user interface 10. Optionally underlying time series ZR1, ZR2 and also further information of significance for the decision can be shown here.

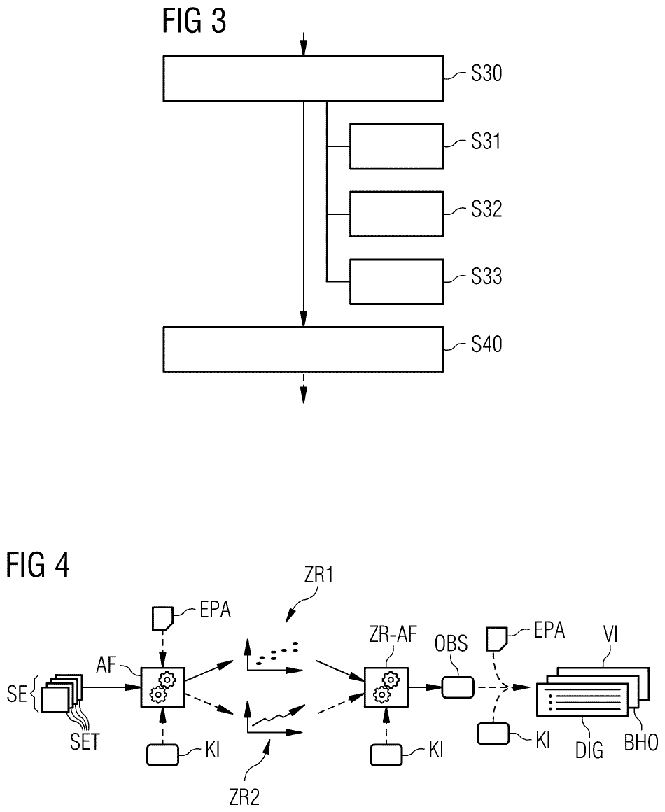

Shown in FIG. 3 is a schematic flow diagram of optional part steps for determination of an observable OBS. The order of the method steps is not restricted either by the order shown or by the numbering selected. Thus the order of the steps can be changed where necessary and individual steps can be left out. Moreover one or more steps, in particular a sequence of steps, can be carried out repeatedly. The part steps shown in FIG. 3 can be carried out within step S30. Shown in FIG. 4 is an associated diagram, which shows examples of the data flows associated with the method shown in FIG. 3.

For a few diseases in can be important to consider time series ZR1, ZR2 not only individually, but in relation to one another. The form of embodiment shown in FIG. 3 has this aspect as its subject.

First of all, in step S31, one or more second time series ZR2 is or are obtained, that are different from the (first) time series ZR1, but which overlap at least partly in time with the first time series ZR1. The second time series ZR2 can in this case likewise be based on image data. As an alternative the second time series ZR2 can be not based on image data. For example the measurement variables NBD-MG reproduced in the second time series ZR2 can be based on laboratory and in particular blood values of the patient. Such second time series ZR2 can for example be taken from the electronic medical record EPA of the patient.

Which second time series ZR2 are to be provided in step S31 can for example be established based on the context information KI.

In accordance with a few examples the second time series ZR2 can also comprise just one individual event. The individual event can for example relate to a therapy and/or a state of health of the patient.

In step S32 a first time series ZR1 is correlated mathematically with a second time series ZR2 in order to establish correlation information KOR. In this case selected first and second time series ZR1, ZR2 can be correlated with one another, wherein a selection can be made from a number of first or second time series ZR1, ZR2, based on the context information KI for example.

As an alternative, first of all, all possible combinations of first and second time series ZR1, ZR2 can be correlated in order to obtain corresponding correlation information KOR or a corresponding observable OBS. Then, with the aid of the correlation information KOR, relevant combinations of time series ZR1, ZR2 can be identified and provided.

Then, from the item or items of correlation information KOR, one or more observables OBS can be established. This can for example comprise a selection with the aid of the correlation information KOR of medically significant combinations of time series ZR1, ZR2. These can be time series combinations such as those showing noticeable temporal correlations.

Step S33 can further comprise a transfer of correlation information KOR in a format that the user N can interpret easily, and which is then provided to the user N as observable OBS. For example the correlation information KOR can itself again be transferred into a time series, which can be displayed together with the underlying time series ZR1, ZR2. An observable OBS established based on the correlation information KOR can further comprise a text that describes the time dependencies encoded in the correlation information KOR for the user N (for example “decrease in the proportion of muscle with simultaneous increase in the AP values in the past two months”). Such a text can for example be provided with an LLM.

Shown in FIG. 5 is a graphical user interface GUI in accordance with one form of embodiment. The graphical user interface GUI can for example be implemented in the user interface 10. The graphical user interface GUI can provide the user N with one or more results of the processing operations described herein.

Time series ZR1, ZR2 can be displayed/shown in one section of the graphical user interface GUI. The time series ZR1, ZR2 can be selected automatically for display, for example based on the observable OBS. As an alternative the time series ZR1, ZR2 can be able to be selected by the user N.

In the example shown in FIG. 5 a time series ZR1 is displayed that is based on an image data-based measurement variable BD-MG. A BCA value is shown here by way of example, which can comprise the individual measurement variables BD-MG relating to a bodily composition of the patient, such as a proportion of body fat or a proportion of muscle, or can comprise a combination of such measurement variables BD-MG. Each measured value in this case can correspond to an imaging examination (for example a whole-body CT scan).

Further shown by way of example in FIG. 5 are three (second) time series ZR2, which are based on non-image data-based measurement variables NBD-MG. In the present example these are the AP, erythrocyte and MCV values of the patient, as can be obtained for example from blood tests of the patient. Individual measured values in such cases can relate to blood tests of a patient. The MCV value stands for mean corpuscular volume. This blood value specifies the mean volume of an individual red blood corpuscle. The value is of importance for example for finding the cause of an anemia. It is determined as part of a blood test, but is not very informative on its own.

A correlation of the time series ZR1 and ZR2 as observable OBS is further shown in the section of the time series ZR1, ZR2. Initially the time series run constantly in a similar way. As from a certain point in time a decrease in the BCA value and an increase in the AP or MCV values is to be observed. This becomes clear in a decrease in the observable OBS, which shows this anticorrelation.

In addition or as an alternative the observable OBS can be provided as a text output. This is shown in a lower left-hand section of the graphical user interface GUI. The text output can be easier to access for the user N than a plot of a correlation. Moreover this text output can be integrated directly into a diagnosis report. In addition possible diagnoses DIG and/or treatment options BHO can be output in further sections of the graphical user interface.

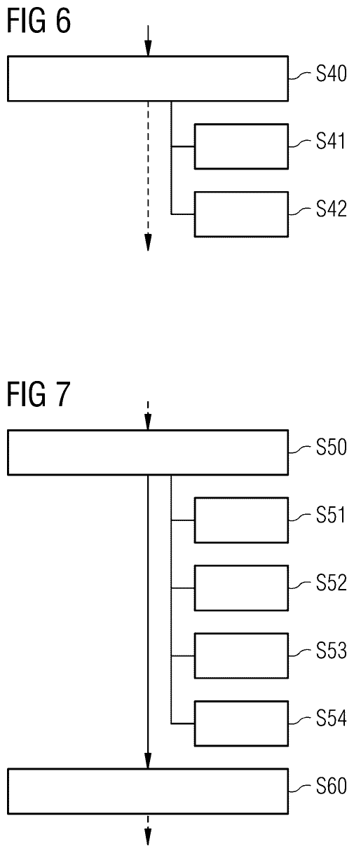

Shown in FIG. 6 is a schematic flow diagram of optional part steps for provision/display of an observable OBS. The order of the method steps is not restricted either by the order shown or by the numbering selected. Thus the order of the steps can be changed where necessary and individual steps can be left out. Moreover one or more steps, in particular a sequence of steps, can be carried out repeatedly. The part steps shown in FIG. 6 can be carried out within step S40.

Depending on the data available, a plurality of observables OBS is possible. In particular a number of time series ZR1, ZR2 open up a plurality of possible combinations for determining correlation information KOR. In order to support the user N in finding relevant observables OBS or time series ZR1, ZR2, there can be provision in step S41 for making a selection with the aid of the observables OBS.

To this end the observables OBS can for example be compared with a threshold value or a specific pattern of observables. This enables a check to be made as to whether the observables OBS for example show a medically relevant state of affairs. Based on the comparison, observables OBS and thus the associated time series ZR1, ZR2 can be selected.

On the basis of the selection in step S41, in step S42, the selected observables OBS and/or time series ZR1, ZR2 can be displayed (cf. FIG. 5).

Shown in FIG. 7 is a schematic flow diagram of optional part steps for determining a medical diagnosis DIG. The order of the method steps is not restricted either by the order shown or by the numbering selected. Thus the order of the steps can be changed where necessary and individual steps can be left out. Moreover one or more steps, in particular a sequence of steps, can be carried out repeatedly. The part steps shown in FIG. 7 can be carried out within step S50.

For determining a medical diagnosis DIG, first of all, in step S51, a disease pattern is identified. To this end disease patterns can be selected from a number of disease patterns to which the observable or observables OBS correspond. In particular the disease pattern that is the most likely based on the observables OBS can be selected.

The number of disease patterns can be preselected based on the context information KI from a plurality of predefined disease patterns. A disease pattern can generally comprise indications and contraindications for a specific disease and thus comprise a diagnosis. At least one of these indications can be an observable OBS described herein.

In step S52 an electronic medical record EPA of the patient is retrieved from the medical information system 40. This can be undertaken for example via an electronic patient identifier such as a patient ID, with which the patient is registered in the medical information system 40.

In step S53 the disease pattern can be reconciled with the electronic medical record. In this case a check can be made as to the extent to which the indications and contraindications of the disease pattern are reflected in the electronic medical record. In accordance with a few examples this can be undertaken by using a transformer network or LLM. This enables indications and contraindications also to be identified in the electronic medical record that have no direct (word-for-word) correspondence in the electronic medical record EPA.

In step S54, based on the reconciliation, a medical diagnosis DIG can be determined. If it can possibly be verified in step S53 that the electronic medical record EPA reflects the indications and contraindications of the disease pattern, it can be concluded that the underlying disease for this disease pattern is present. This can be provided as the diagnosis DIG. Conversely, if the indications and contraindications are not present, the disease can be excluded. This too can be provided as the medical diagnosis DIG.

Optionally step S51 and step S53 can again follow on from step S54 in order to reconcile further disease patterns with the electronic medical record EPA. In such cases, in step S51, based on the observable OBS, the next most likely disease pattern can be selected in each case.

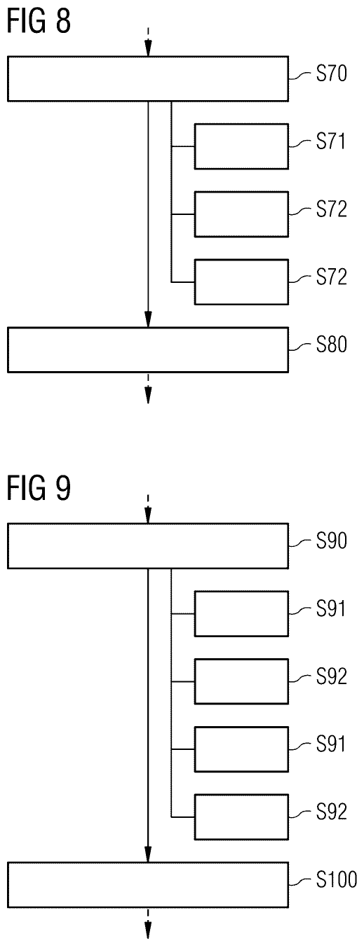

Shown in FIG. 8 is a schematic flow diagram of optional part steps for finding comparison patients. The order of the method steps is not restricted either by the order shown or by the numbering selected. Thus the order of the steps can be changed where necessary and individual steps can be left out. Moreover one or more steps, in particular a sequence of steps, can be carried out repeatedly. The part steps shown in FIG. 8 can be carried out within step S70.

Step S70 handles the establishing of comparison patients from a number of reference patients. The reference patients can each be registered in the medical information system 40. An electronic medical record EPA can be set up for each reference patient in the medical information system 40. Observables that correspond with the observables OBS for the patient can be held in the electronic medical records EPA. As an alternative such corresponding observables can be created for the reference patients by a similar processing as for the patient.

In step S71 there can be a comparison of the observable OBS with corresponding observables of reference patients. In this step one or more observables OBS can be included for the comparison. Optionally the reference patients can be preselected in this case from a larger group of patients registered in the medical information system 40 based on the context information KI.

Then, in step S72, based on step S71, those reference patients can be identified as comparison patients who, based on the observable OBS, have a certain similarity to the patient. This can in particular be such reference patients as show similar corresponding observables to the observable OBS.

The comparison patients can be notified to the user N. In addition, in step S73 there can be provision for establishing information about the selected comparison patient (comparison information VI). The comparison information VI can be extracted from the respective electronic medical records EPA of the comparison patients. The comparison information VI can for example comprise a diagnosis DIG or a treatment option BHO. In addition the comparison information VI can comprise one or more diagnostic reports of the comparison patients.

Shown in FIG. 9 is a schematic flow diagram of optional part steps for providing/displaying a treatment option BHO. The order of the method steps is not restricted either by the order shown or by the numbering selected. Thus the order of the steps can be changed where necessary and individual steps can be left out. Moreover one or more steps, in particular a sequence of steps, can be carried out repeatedly. The part steps shown in FIG. 9 can be carried out within step S90.

First of all, in step S91, based the observable or observables OBS, a disease pattern can be selected. In this case the method can basically be the same as that in step S51. The disease patterns in this form of embodiment are each associated with at least one treatment option BHO. The treatment options BHO can be prespecified for example based on a medical guideline for the disease underlying the disease pattern. The treatment options BHO can each be associated with further indicators that specify whether the treatment options BHO are suitable for the respective patient. Such indicators can for example comprise allergies to specific medicaments or an age restriction and the like.

In step S92 an electronic medical record EPA of the patient can be retrieved from the medical information system 40. In this case the procedure can basically be the same as in step S52.

In step S93 the disease pattern is reconciled with the electronic medical record EPA. In this case the procedure can basically be the same as in step S53.

Based on the reconciliation in step S93, in step S94 a treatment option BHO can be determined. If it can possibly be verified in step S93 that the disease pattern is present, the associated treatment option BHO can be provided. In order to safeguard the treatment option BHO, for indications or contraindications of the disease pattern or indicators of the treatment option BHO for which no information is to be found in the electronic medical record EPA, further information about the patient is retrieved automatically (for example for user N) and further examinations of the patient are proposed or initiated in the medical information system 40.

While exemplary embodiments have been described in detail, in particular with reference to the figures, it should be pointed out that a plurality of variations is possible. Moreover it should be pointed out that the exemplary embodiments merely involve examples, which are not intended in any way to restrict the scope of protection, the application and the structure. Instead the person skilled in the art is given a guideline by the preceding description for the implementation of at least one exemplary embodiment, wherein diverse variations, in particular alternative or additional features and/or variation of the function and/or arrangement of the elements described can be made as the person skilled in the art wishes without, in doing so, departing from the subject matter defined in each case in the appended claims as well as its legal equivalent and/or leaving its area of protection.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A computer-implemented method for providing an observable indicating a medical diagnosis, the computer-implemented method comprising:
obtaining a medical image data series of a patient, wherein the medical image data series has a number of medical image datasets, which have each been recorded over a first period of time at different points in time;
extracting a first time series from the medical image data series;
determining the observable based on the first time series; and
providing the observable.

2. The computer-implemented method as claimed in claim 1, wherein the determining the observable comprises:
obtaining a second time series of a measurement variable associated with the patient, said second time series covering a second period of time, which at least partly overlaps with the first period of time; and
determining the observable additionally based on the second time series.

3. The computer-implemented method as claimed in claim 2, wherein the determining of the observable comprises:
determining correlation information based on the first time series and the second time series, wherein the correlation information specifies a measure for a temporal correlation between the first time series and the second time series; and
establishing the observable based on the correlation information.

4. The computer-implemented method as claimed in claim 2, wherein the measurement variable of the second time series is not based on a medical imaging.

5. The computer-implemented method as claimed in claim 4, wherein the measurement variable of the second time series includes one or more blood values of the patient.

6. The computer-implemented method as claimed in claim 1, wherein the determining the observable comprises:
obtaining an individual event that lies within the first period of time, the individual event being associated with the patient;
determining correlation information based on the first time series and the individual event, wherein the correlation information specifies a measure of a temporal correlation between the first time series and the individual event; and
establishing the observable based on the correlation information.

7. The computer-implemented method as claimed in claim 1, further comprising:
obtaining context information concerning a clinical picture of the patient, wherein
the extracting includes selecting an image data-based measurement variable from a number of different image data-based measurement variables based on the context information, and
the first time series is a time series of the selected image data-based measurement variable.

8. The computer-implemented method as claimed in claim 1, wherein each medical image dataset includes a recording of an entire area of the body of the patient or a recording of the entire body of the patient.

9. The computer-implemented method as claimed in claim 1, wherein a measurement variable of the first time series is selected from:
an analysis value of a bodily composition, BCA value, including at least one of a proportion of muscle or a proportion of body fat,
a size of a lesion, or
a tumor burden in a part of the body or an area of the body of the patient or in the entire body of the patient.

10. The computer-implemented method as claimed in claim 1, wherein the providing comprises:
comparing the observable with an observable pattern; and
displaying the first time series or the observable based on the comparison.

11. The computer-implemented method as claimed in claim 1, further comprising:
determining a medical diagnosis based on the observable; and
providing the medical diagnosis.

12. The computer-implemented method as claimed in claim 11, wherein the determining the medical diagnosis comprises:
selecting a disease pattern from a number of disease pattern choices based on the observable;
retrieving an electronic medical record of the patient from a database;
reconciling the disease pattern with entries in the electronic medical record; and
determining the medical diagnosis based on the reconciliation.

13. The computer-implemented method as claimed in claim 1, further comprising:
comparing the observable with corresponding observables of a number of reference patients that are different from the patient;
selecting a comparison patient from the number of reference patients based on the comparing; and
providing information about the comparison patient.

14. The computer-implemented method as claimed in claim 1, further comprising:
determining a treatment option based on the observable; and
providing the treatment option.

15. A system for providing an observable indicating a medical diagnosis, the system comprising:
an interface configured to obtain an image data series of a patient, wherein the image data series has a number of medical image datasets, which have been recorded over a period of time at different points in time; and
a computing device configured to
extract a first time series from the image data series,
determine the observable based on the first time series, and
provide the observable via the interface.

16. A non-transitory computer program product comprising a program that is loadable into a memory of a programmable processing unit, the program including program instructions that cause the programmable processing unit to carry out the method as claimed in claim 1 when the program is executed at the programmable processing unit.

17. A non-transitory computer-readable medium storing readable and executable program sections that, when executed by a computing device of a system, cause the system to perform the method as claimed in claim 1.

18. The computer-implemented method as claimed in claim 4, wherein the measurement variable of the second time series includes one or more laboratory values of the patient.

19. The computer-implemented method as claimed in claim 2, wherein the determining the observable comprises:
obtaining an individual event that lies within the first period of time, the individual event being associated with the patient;
determining correlation information based on the first time series and the individual event, wherein the correlation information specifies a measure of a temporal correlation between the first time series and the individual event; and
establishing the observable based on the correlation information.

20. The computer-implemented method as claimed in claim 6, further comprising:
obtaining context information concerning a clinical picture of the patient, wherein
the extracting includes selecting an image data-based measurement variable from a number of different image data-based measurement variables based on the context information, and
the first time series is a time series of the selected image data-based measurement variable.
